(19) United States
(12) Patent Application Publication
(10) Pub. No.: US 2012/0203049 A1
(43) Pub. Date: Aug. 9, 2012

(54) HEAT EXCHANGE DEVICE AND A METHOD OF MANUFACTURING THE SAME

(75) Inventors: Frank D. McCarthy, Wayne, NJ (US); Steve De Haan, Wayne, NJ (US)

(73) Assignee: LUMMUS TECHNOLOGY INC., Bloomfield, NJ (US)

(21) Appl. No.: 13/498,834

(22) PCT Filed: Feb. 8, 2011
(86) PCT No.: PCT/US11/24008

§ 371 Date: Mar. 28, 2012

(51) Int. Cl. C07C 4/04; F28F 13/12; F28F 7/00; B23P 15/26

(52) U.S. Cl. 585/648; 29/890.03; 165/109.1; 165/53

(57) ABSTRACT

A method of manufacturing a heat exchange device having at least one heat exchange tube is disclosed. The method includes: determining a peak heat flux area of the at least one heat exchange tube; and disposing in the at least one heat exchange tube an flow enhancement device for creating a desirable flow pattern in a process fluid flowing through the at least one heat exchange tube; wherein the flow enhancement device is disposed in the at least one heat exchange tube upstream of or at the determined peak heat flux area of the at least one heat exchange tube.

### FIELD OF THE DISCLOSURE

Embodiments disclosed herein relate generally to the cracking (pyrolysis) of hydrocarbons, and to a heat exchanger and processes for effecting the cracking of the hydrocarbons at higher selectivity and longer run times.

### BACKGROUND

Heat exchangers are used in a variety of applications to heat or cool fluids and/or gases, typically by means of indirect heat transfer through different intervening layers of heat exchange tubes. For example, heat exchangers may be used in air conditioning systems, refrigeration systems, radiators, or other similar systems used for heating or cooling, as well as in processing systems such as geothermal energy production. Heat exchangers are particularly useful in petroleum hydrocarbon processing as a means to facilitate processing reactions using less energy. Delayed cokers, vacuum heaters, and cracking heaters are heat exchange devices commonly used in petroleum hydrocarbon processing.

Numerous configurations for heat exchangers are known and used in the art. For example, a common configuration for heat exchangers is a shell and tube heat exchanger, which includes a cylindrical shell housing a bundle of parallel pipes. A first fluid passes through the pipes while a second fluid passes through the shell, around the pipes, such that heat exchanges between the two fluids. In some shell and tube configurations, baffles are arranged throughout the shell and around the tubes so that the second fluid flows in a particular direction to optimize heat transfer. Other configurations for heat exchangers include fired heaters, double-pipe, plate, plate-fin, plate-and-frame, spiral, air-cooled, and coil heat exchangers, for example. Embodiments disclosed herein relate generally to heat exchange tubes used within a heat exchange device.

Generally, the heat transfer rate of a heat exchange tube may be represented by the convection equation: Q=UAΔT, wherein Q is the heat transferred per unit time, A is the area available for the flow of heat, ΔT is the temperature difference for the entire heat exchanger, and U is the overall heat transfer coefficient based on the area available for the flow of heat, A.

It is well known in the art that the rate of heat transfer, Q, may be increased by increasing the area available for the flow of heat, A. Thus, a commonly used method for increasing the amount of heat transfer is to increase the amount of surface area in the heat exchange tube. One such method involves using multiple small diameter heat exchange tubes rather than a single larger diameter heat exchange tube. Other methods of increasing the heat transfer area of the tube wall include adding a variety of patterns, fins, channels, ridges, grooves, flow enhancement devices, etc. along the tube wall. Such surface variations may also indirectly increase the heat transfer area by creating turbulence in the fluid flow. Specifically, turbulent fluid flow allows for a higher percentage of fluid to contact the tube wall, thereby increasing the heat transfer rate.

For example, U.S. Pat. No. 3,071,159 describes a heat exchanger tube having an elongated body with several members extending there from, inserted within the heat exchanger tube, such that fluid is channeled close to the wall of the heat exchanger tube and the fluid has a turbulent flow. Other heat exchange tubes with patterns, including fins, ribs, channels, grooves, bulges, and/or inserts along the tube wall are described in, for example, U.S. Pat. No. 3,885,622, U.S. Pat. No. 4,438,808, U.S. Pat. No. 5,203,404, U.S. Pat. No. 5,236,045, U.S. Pat. No. 5,332,034, U.S. Pat. No. 5,333,682, U.S. Pat. No. 5,950,718, U.S. Pat. No. 6,250,340, U.S. Pat. No. 6,308,775, U.S. Pat. No. 6,470,964, U.S. Pat. No. 6,644,358, and U.S. Pat. No. 6,719,953.

It is also known in the art that the heat transfer coefficient, U, is largely a function of the thermal conductivity of the heat exchange tube material, the geometric configuration of the heat exchange tube, and flow conditions of fluid within and around the heat exchange tube. These variables are frequently interrelated, and thus, they may be considered in conjunction with one another. In particular, the geometric configuration of the heat exchange tube affects flow conditions. Poor flow conditions may result in fouling, which is the build up of undesirable deposits on the walls of the heat exchange tube. Increased amounts of fouling impede the thermal conductivity of the heat exchange tube. Thus, heat exchange tubes are often geometrically configured to increase fluid flow velocity and encourage turbulence in the fluid flow as a way to break up and prevent fouling.

In addition to impeding the thermal conductivity of the heat exchange tube, an increased amount of fouling may also create a pressure drop throughout the tube. Pressure drops in heat exchange tubes may result in increased processing costs required to restore the pressure within the tube. Furthermore, pressure drops may limit the fluid flow rate, thereby reducing the heat transfer rate.

As described above, adding various patterns and inserts to a heat exchanger tube wall are commonly implemented methods of increasing the heat transfer area and providing a more turbulent fluid flow, and thereby increasing the heat transfer rate of a heat exchanger tube. However, the addition of such mechanical modifications often requires higher material costs, expensive manufacturing procedures, and increased energy costs (including heating more tube material). Additionally, inserts, fins, and the like may cause spalling in certain applications, such as in cracking heaters or delayed cokers.

Ethylene is produced worldwide in large quantities, primarily for use as a chemical building block for other materials. Ethylene emerged as a large volume intermediate product in the 1940s when oil and chemical producing companies began separating ethylene from refinery waste gas or producing ethylene from ethane obtained from refinery byproduct streams and from natural gas.

Most ethylene is produced by thermal cracking of ethylene with steam. Hydrocarbon cracking generally occurs in fired tubular reactors in the radiant section of the furnace. In a convection section, a hydrocarbon stream may be preheated by heat exchange with flue gas from the furnace burners, and further heated using steam to raise the temperature to incipient cracking temperatures, typically 500-680° C. depending on the feedstock.

After preheating, the feed stream enters the radiant section of the furnace in tubes referred to herein as radiant coils. It should be understood that the method described and claimed can be performed in ethylene cracking furnaces having any type of radiant coils. In the radiant coils, the hydrocarbon stream is heated under controlled residence time, temperature and pressure, typically to temperatures in the range of about 780-895° C. for a short time period. The hydrocarbons in the feed stream are cracked into smaller molecules, including ethylene and other olefins. The cracked products are then separated into the desired products using various separation or chemical-treatment steps.

Various byproducts are formed during the cracking process. Among the byproducts formed is coke, which can deposit on the surfaces of the tubes in the furnace. Coking of the radiant coils reduces heat transfer and the efficiency of the cracking process as well as increasing the coil pressure drop. Therefore, periodically, a limit is reached and decoking of the furnace coils is required.

As decoking causes a disruption in production and thermal cycling of equipment, very long run lengths are desirable. Various methods have been devised to extend radiant coil run lengths. These include chemical additives, coated radiant tubes, mechanical devices that change flow patterns, as well as other methods.

The mechanical devices or more generally radiant coil flow enhancement devices have been most successful in extending run lengths. These devices increase run length by changing flow patterns to a “desirable flow pattern” in the radiant tube in order to: increase heat transfer rates; reduce the thickness of the stagnant film along the tube wall and thus limiting reactions that cause coking of the tube; and improve the radial temperature profile within the radiant tube.

However, these devices have a significant drawback. Use of these devices causes an increase in radiant coil pressure drop, which negatively impacts the yield of valuable cracking products. This loss of yield has a significant impact on operating economics and is therefore a significant limitation.

### SUMMARY OF THE CLAIMED EMBODIMENTS

The intent of the present invention is to overcome the limitation caused by loss of yield by locating the chosen radiant coil flow enhancement device(s) in a strategic position(s) in the radiant coil. Until now many radiant coil flow enhancement devices have been used throughout the coil or at least in the entire length of one pass of the coil. Others have been specifically located, however, the location has been arbitrary or standard. This invention seeks to locate these devices strategically to maximize their impact and minimize the additional pressure drop created.

In one aspect, embodiments disclosed herein relate to a method of manufacturing a heat exchange device having at least one heat exchange tube, comprising:
determining a peak heat flux area of the at least one heat exchange tube; and
disposing in the at least one heat exchange tube an flow enhancement device for creating a desirable flow pattern in a process fluid flowing through the at least one heat exchange tube;
wherein the flow enhancement device is disposed in the at least one heat exchange tube upstream of or at the determined peak heat flux area of the at least one heat exchange tube.

In another aspect, embodiments disclosed herein relate to a method of retrofitting a heat exchange device having at least one heat exchange tube, comprising:
determining a peak heat flux area of the at least one heat exchange tube; and
replacing at least a portion of the at least one heat exchange tube upstream of the determined peak heat flux area with a flow enhancement device for creating a desirable flow pattern in a process fluid flowing through the at least one heat exchange tube.

In another aspect, embodiments disclosed herein relate to a heat exchange device, comprising:
at least one heat exchange tube; and
a flow enhancement device disposed in the at least one heat exchange tube for creating a desirable flow pattern in a process fluid flowing through the at least one heat exchange tube;
wherein the flow enhancement device is disposed in the at least one heat exchange tube upstream of or at a determined peak heat flux area of the at least one heat exchange tube.

In another aspect, embodiments disclosed herein relate to a process for producing olefins, the process comprising:
passing a hydrocarbon through a heat exchange tube in a radiant heating chamber at conditions to effect pyrolysis of the hydrocarbon, the heat exchange tube having an flow enhancement device disposed therein for creating a desirable flow pattern of the hydrocarbon flowing through the heat exchange tube;
wherein the flow enhancement device was selectively disposed in the at least one heat exchange tube upstream of or at a determined peak heat flux area of the at least one heat exchange tube.

Other aspects and advantages will be apparent from the following description and the appended claims.

### BRIEF DESCRIPTION OF DRAWINGS

FIG. 1 illustrates a method for manufacturing a heat exchange device according to embodiments disclosed herein.

FIG. 2 illustrates a simplified cross-section of a typical prior art pyrolysis heater.

FIG. 3 is a graph illustrating a surface heat flux profile throughout the elevation of a pyrolysis heater.

FIG. 4 is a graph illustrating a surface metal temperature profile throughout the elevation of a pyrolysis heater.

FIG. 5 illustrates a method for retrofitting a heat exchange device according to embodiments disclosed herein.

FIG. 6 illustrates a radiant coil of a heat exchange device according to embodiments disclosed herein.

FIG. 7 illustrates a method for manufacturing a heat exchange device according to embodiments disclosed herein.

FIG. 8 illustrates a method for manufacturing a heat exchange device according to embodiments disclosed herein.

FIGS. 9A and 9B illustrates a radiant coil insert useful in embodiments disclosed herein.

### DETAILED DESCRIPTION

In one aspect, embodiments herein relate to the cracking (pyrolysis) of hydrocarbons. In other aspects, embodiments disclosed herein relate to a heat exchanger and processes for effecting the cracking of the hydrocarbons at higher selectivity and longer run times.

Radiant coil flow enhancement devices, as mentioned above, are used to promote desirable flow profiles within the radiant coil to improve heat transfer, reduce coking, and enhance radial temperature profiles. Such devices are currently placed throughout the entire length of the radiant coil or distributed throughout the length of the coil, such as at a given length interval.

It has now been surprisingly discovered that selective placement of radiant coil flow enhancement devices at a location upstream of or at a peak heat flux area of a radiant coil or a radiant coil pass may provide for one or more of the following as compared to prior radiant coil flow enhancement device placement methods: i) an increased or maximized selectivity and yields to valuable olefins; ii) an extended heater run length and capacity; iii) a minimized or decreased number of flow enhancement devices used in a radiant coil; and iv) a minimized or decreased pressure drop through a radiant coil.

As used herein, placement “upstream” of or at a peak heat flux area refers to locating a flow enhancement device in a radiant coil tube such that the flow profile resulting from the device extends through the peak heat flux area of the radiant coil. One skilled in the art would recognize that the flow pattern induced by the radiant coil flow enhancement devices exists in the device and extends only for a limited distance after the end of the device, and merely placing a flow enhancement device in a coil may not result in the desired flow pattern extending through the peak heat flux area. The placement of the device relative to the peak heat flux area is selected, according to embodiments disclosed herein, such that the desired flow zone extends through the peak heat flux area, and such placement may depend upon a number of factors including the type and size of the radiant coil flow enhancement device (axial length of the flow enhancement device, number of flow passages through the flow enhancement device, twist angle(s), etc.), the flow rate of hydrocarbons and/or steam through the coil, and coil diameter, among others.

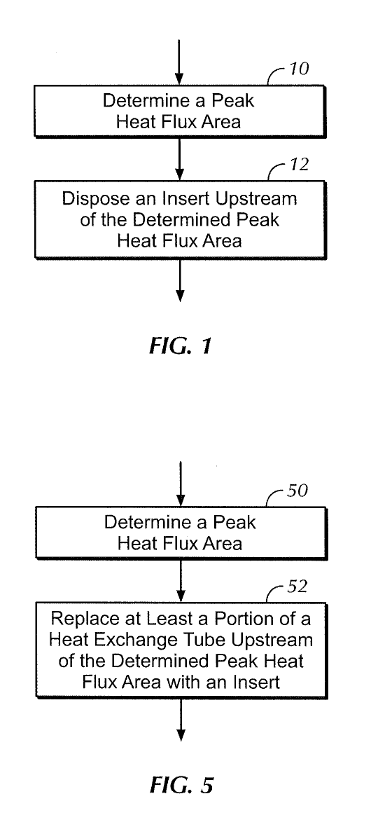

Referring now to FIG. 1, a method for manufacturing a heat exchange device having at least one heat exchange tube is illustrated. In step 10, for a given heat exchange device or heat exchanger design, a heat flux profile for the heat exchange device is determined. For example, a furnace (a type of heat exchange device useful for pyrolysis of hydrocarbons) may have a particular design, including a number of burners, burner location, types of burners, etc. The furnace will thus provide a particular flame profile (radiant heat) and a combustion gas circulation profile (convective heat) based on the furnace design, allowing for the determination of the heat flux profile for the furnace. Due to the radiant and convective driving forces, the heat flux profile will vary over the length or height of the furnace, in virtually all instances, and the determined profile will have one or more peak heat flux elevations (i.e., an elevation in the furnace where the heat flux is at a maximum). In step 12, based on the determined heat flux profile, a flow enhancement device may be disposed in the at least one heat exchange tube upstream of or at the determined peak heat flux area to promote a desirable flow pattern through the determined peak heat flux area.

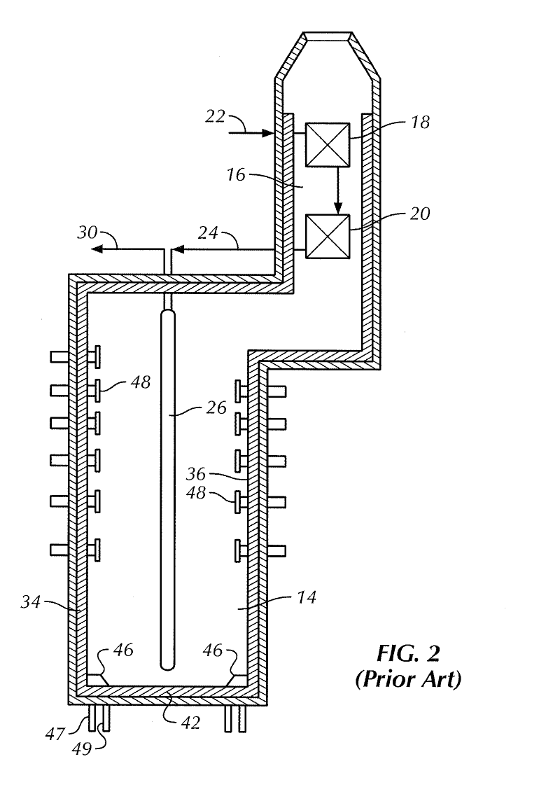

As an example of the method for manufacturing a heat exchange device having at least one heat exchange tube, reference is made to FIGS. 1-3 of U.S. Pat. No. 6,685,893, illustrated herein as FIGS. 2-4. A cross-section of a typical prior art pyrolysis heater is illustrated in FIG. 2. The heater has a radiant heating zone 14 and a convection heating zone 16. Located in the convection heating zone 16 are the heat exchange surfaces 18 and 20 which in this case are illustrated for preheating the hydrocarbon feed 22. This zone may also contain heat exchange surface for producing steam. The preheated feed from the convection zone is fed at 24 to the heating coil generally designated 26 located in the radiant heating zone 14. The cracked product from the heating coil 26 exits at 30. The heating coils may be any desired configuration including vertical and horizontal coils as are common in the industry.

The radiant heating zone 14 comprises walls designated 34 and 36 and floor or hearth 42. Mounted on the floor are the vertically firing hearth burners 46 which are directed up along the walls and which are supplied with air 47 and fuel 49. Usually mounted in the walls are the wall burners 48 which are radiant-type burners designed to produce flat flame patterns which are spread across the walls to avoid flame impingement on the coil tubes.

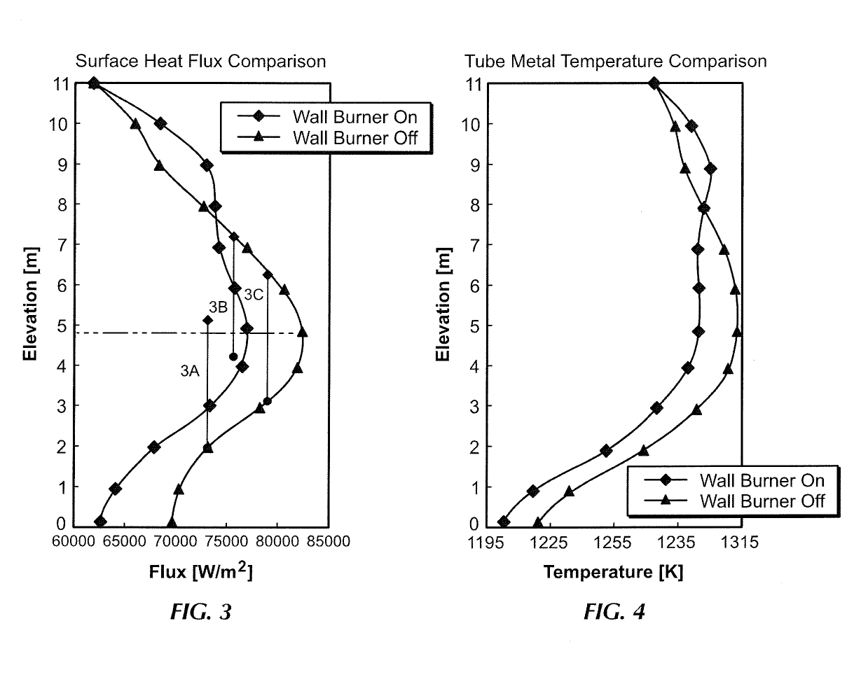

In step 10 of the method of FIG. 1, the heat flux profile for the heater is determined. FIG. 3 shows results of step 10, illustrating a typical surface heat flux profile for the heater as illustrated in FIG. 2 for two operational modes, with both the hearth burners and wall burners being on in one case and with the hearth burners being on and the wall burners being off in the other case. FIG. 4 shows the tube metal temperature determined under the same conditions. These figures show low heat flux and low metal temperatures in both the lower part of the firebox and the upper part of the firebox and show a large difference between the minimum and maximum of the temperature or the heat flux.

The peak heat flux for both operational modes is determined to occur at an elevation of approximately 5 meters. In step 12, a radiant coil flow enhancement device may be disposed in one or more heat exchange tubes of coil 26 upstream of or at the peak heat flux elevation, above or below the 5 meter elevation depending upon the flow direction, such that the desirable flow zone generated by the flow enhancement device extends through the peak heat flux area of the one or more tubes or tube passes.

Referring now to FIG. 5, a method for retrofitting an existing heat exchange device having at least one heat exchange tube is illustrated. In step 50, for a given heat exchange device or heat exchanger design, a heat flux profile for the heat exchange device is determined. For example, a furnace (a type of heat exchange device useful for pyrolysis of hydrocarbons) may have a particular design, including a number of burners, burner location, types of burners, etc. The furnace will thus provide a particular flame profile (radiant heat) and a combustion gas circulation profile (convective heat) based on the furnace design, allowing for the determination of the heat flux profile for the furnace. Due to the radiant and convective driving forces, the heat flux profile will vary over the length or height of the furnace, in virtually all instances, and the determined profile will have one or more peak heat flux elevations (i.e., an elevation in the furnace where the heat flux is at a maximum). In step 52, based on the determined heat flux profile, at least a portion of at least one heat exchange tube upstream of or at the determined peak heat flux area is replaced with a flow enhancement device for creating the desired flow pattern.

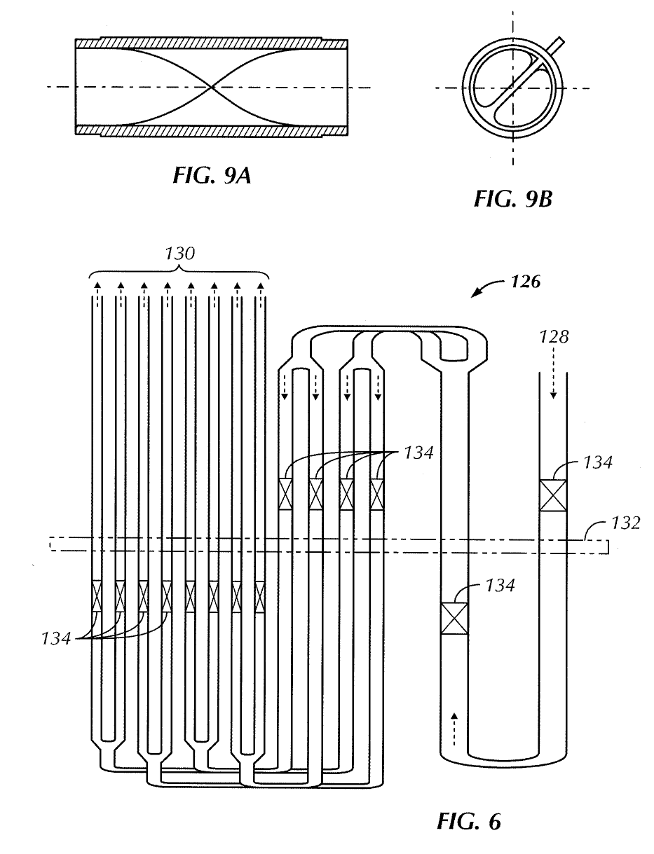

The heat exchange coil or coils disposed in heat exchange device may make multiple passes through the heat transfer area. For example, a heating coil 26, as illustrated in the furnace of FIG. 2, may make one or more passes through radiant heating zone 14. FIG. 6 illustrates a heat exchange coil 126 having four passes through the radiant heating zone, for example, where the hydrocarbon flow enters the first heating tube at 128 and traverses through the multiple passes and exits the coil at 130. The heat exchange coil 126 may be disposed in a furnace having a determined peak heat flux area corresponding to that illustrated by area 132. Radiant coil flow enhancement device may be disposed in one, two, or more of the tube passes through the heat exchange column, where the flow enhancement device(s) are disposed upstream of or at the determined peak heat flux area 132 according to embodiments disclosed herein. As illustrated in FIG. 6, radiant coil flow enhancement device 134 are disposed in each of the tube passes upstream of or at the peak heat flux area as based on the indicated flow direction.

As mentioned above, the flow pattern induced by the radiant coil flow enhancement device only extends for a limited distance, and the placement of the flow enhancement device relative to the peak heat flux area may be selected, according to embodiments disclosed herein, such that the desirable flow zone extends through the peak heat flux area. The placement may depend upon a number of factors including the type and size of the radiant coil flow enhancement device (axial length of the flow enhancement device, number of flow passages through the flow enhancement device, twist angle(s), etc.), the flow rate of hydrocarbons and/or steam through the coil, and coil diameter, among others.

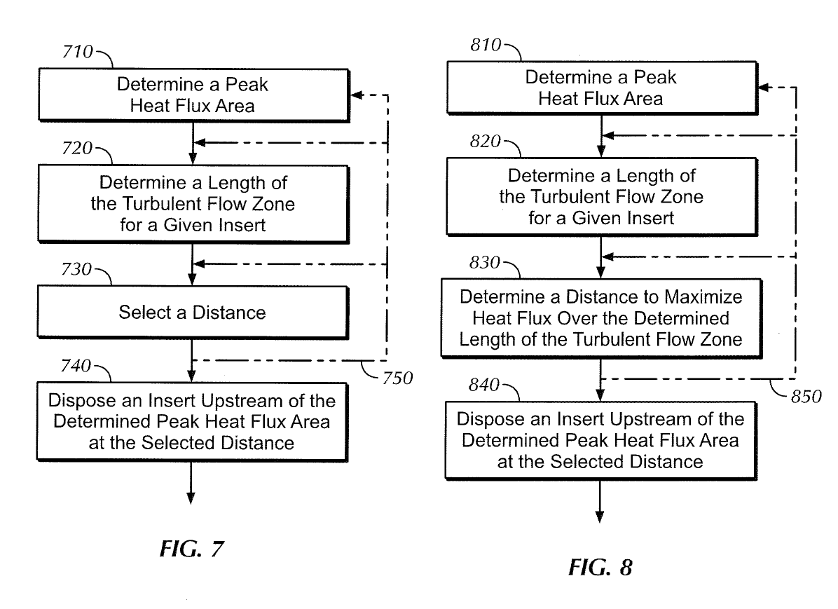

In some embodiments, the method of manufacturing or retrofitting a heat exchange device may include additional steps to select a suitable or optimal location of the flow enhancement device. Referring now to FIG. 7, a method for manufacturing a heat exchange device having at least one heat exchange tube is illustrated. Similar to the method of FIG. 1, in step 710, for a given heat exchange device or heat exchanger design, a heat flux profile for the heat exchange device is determined along with the peak heat flux area. In step 720, a length of the desirable flow pattern zone resulting from placement of a given flow enhancement device in a heat exchange tube may be determined. This length may then be used in step 730 to select a distance upstream of the determined peak heat flux area to dispose the flow enhancement device in the at least one heat exchange tube such that the desirable flow pattern zone extends through the peak heat flux area. The flow enhancement device may then be disposed at the selected distance upstream of or at the determined peak heat flux area in step 740.

As noted above, the length of the desirable flow pattern zone may vary based upon flow enhancement device design, among other factors. Referring again to FIG. 3, assuming upward fluid flow, a flow enhancement device having a determined desirable flow pattern zone length of 3 meters may be located anywhere from about 2 meters to about 4.5 meters to result in a desirable flow pattern zone extending through the peak heat flux area, as illustrated by lines 3A and 3B, respectively. The selected distance may depend upon tube location and design, such as having to account for bends in the coil and coil support structures, among other factors.

While locating a flow enhancement device within this range may result in acceptable performance improvements, it may additionally be desired to maximize the heat flux over the determined length of the desirable flow pattern zone. Referring now to FIG. 8, in step 810, for a given heat exchange device or heat exchanger design, a heat flux profile for the heat exchange device is determined along with the peak heat flux area. In step 820, a length of the desirable flow pattern zone resulting from placement of a given flow enhancement device in a heat exchange tube may be determined. This length may then be used in step 830 to determine a distance upstream of the determined peak heat flux area to dispose the flow enhancement device in the at least one heat exchange tube to maximize the heat flux over the determined length of the desirable flow pattern zone. The flow enhancement device may then be disposed at the determined distance upstream of or at the determined peak heat flux area in step 840.

Referring again to FIG. 3, and again assuming upward fluid flow, a flow enhancement device having a determined desirable flow pattern zone length of 3 meters may be located anywhere from about 2 meters to about 4.5 meters. Determination of the distance to maximize heat flux in step 830 may indicate that placement of the flow enhancement device at an elevation of approximately 3 meters may maximize the heat flux over the determined length of the desirable flow pattern zone. Although not illustrated, a similar analysis may be performed for flow enhancement device having different determined desirable flow pattern zone lengths.

It may be desired to maximize the heat flux in some embodiments, as described above. It is additionally noted that the performance of a heat exchange device may not rest solely with the heat transfer attained. For example, performance of a furnace used for pyrolysis of hydrocarbons may be scrutinized based on various operating parameters such as pressure drop through the heating coil(s), selectivity and/or yield to a reaction product such as olefins, fouling or coking rates of the radiant surfaces (heater run length before shutting down), and cost (number of flow enhancement devices, for example), among others. Referring to FIGS. 7 and 8, one or more of steps 710, 720, and 730 (810, 820, and 830) may be repeated through iterations (750, 850) to optimize one or more of the heat flux over the length of the desirable flow pattern zone, the length of the desirable flow pattern zone, a design of the flow enhancement device, and an operating parameter of the heat exchange device.

Flow enhancement devices, as mentioned above, may vary in design. Flow enhancement devices may divide the fluid flow into two, three, four, or more passages, can have a twisted angle of the flow enhancement device baffle in the range from about 100° to 360° or more, and may vary in length from about 100 mm to the full tube length in some embodiments, and from about 200 mm to the full tube length in other embodiments. In other embodiments, the length of the flow enhancement device may be in the range from about 100 mm to about 1000 mm; or from about 200 mm to about 500 mm in yet other embodiments. The thickness of the baffle may be approximately the same as the coil tube in some embodiments. Preferably, the baffle and the surface of the coil piece holding it in place has the shape of a concave circular arc or a similar shape to minimize eddy formation through the passages, reducing flow resistance and pressure drop. The flow enhancement devices may be made, for example, by means of smelting the raw material in the vacuum condition and precision casting, where the flow enhancement device mold is inserted into the coil piece and the required amount of alloy is poured into the mold to form the baffle and the mold burns away in the process. The flow enhancement device can be installed by a cut-and-paste approach into new or existing tubes. Alternately the flow enhancement devices can be formed by adding a weld bead or other helical fin to a standard bare tube. This weld bead can be continuous or discontinuous and may or may not extend the length of the radiant tube.

One example of a radiant coil flow enhancement device is illustrated in FIGS. 9A (profile view) and 9B (end view). The radiant coil flow enhancement device illustrated divides the fluid flow into two flow paths traversing the length of the flow enhancement device. The coil includes a baffle having a twisted angle of approximately 180°.

As mentioned above, flow enhancement devices may be useful in furnaces used for the pyrolysis (cracking) of hydrocarbon feedstocks. The hydrocarbon feedstock may be any one of a wide variety of typical cracking feedstocks such as methane, ethane, propane, butane, mixtures of these gases, naphthas, gas oils, etc. The product stream contains a variety of components the concentration of which is dependent in part upon the feed selected. In a conventional pyrolysis process, vaporized feedstock is fed together with dilution steam to a tubular reactor located within the fired heater. The quantity of dilution steam required is dependent upon the feedstock selected; lighter feedstocks such as ethane require lower steam (0.2 lb./lb. feed), while heavier feedstocks such as naphtha and gas oils require steam/feed ratios of 0.5 to 1.0. The dilution steam has the dual function of lowering the partial pressure of the hydrocarbon and reducing the carburization rate of the pyrolysis coils.

In a typical pyrolysis process, the steam/hydrocarbon feed mixture is preheated to a temperature just below the onset of the cracking reaction, such as about 650° C. This preheat occurs in the convection section of the heater. The mix then passes to the radiant section where the pyrolysis reactions occur. Generally the residence time in the pyrolysis coil is in the range of 0.05 to 2 seconds and outlet temperatures for the reaction are on the order of 700° C. to 1200° C. The reactions that result in the transformation of saturated hydrocarbons to olefins are highly endothermic, thus requiring high levels of heat input. This heat input must occur at the elevated reaction temperatures. It is generally recognized in the industry that for most feedstocks, and especially for heavier feedstocks such as naphtha, shorter residence times will lead to higher selectivity to ethylene and propylene as secondary degradation reactions will be reduced. Further it is recognized that the lower the partial pressure of the hydrocarbon within the reaction environment, the higher the selectivity.

In pyrolysis heaters, the rate of fouling (coking) is set by the metal temperature and its influence on the coking reactions that occur within the inner film of the process coil. The lower the metal temperature, the lower the rates of coking. The coke formed on the inner surface of the coil creates a thermal resistance to heat transfer. In order for the same process heat input to be obtained as the coil fouls, furnace firing must increase and outside metal temperature must increase to compensate for the resistance of the coke layer.

The peak heat flux areas of the furnace thus limit the overall performance of the furnace and the cracking process due to fouling/coking at the high metal temperatures. Embodiments disclosed herein, disposing flow enhancement devices at selected or determined locations within the coil may thus provide numerous benefits. The flow patterns induced by the flow enhancement devices through the peak heat flux area may decrease or minimize fouling through the portion of the coil having the highest metal temperature. As a result of the strategic placement of the flow enhancement devices, the reduced fouling rate may allow for extended run times. Additionally, disposing flow enhancement devices in the coil in limited locations, such as only upstream of or at peak heat flux area(s) and not throughout the entirety of the coil, pressure drop through the coil may be decreased or minimized, thus improving one or more of selectivity, yield, and capacity. The longer run times, improved selectivity, improved yield and/or improved capacity attainable according to embodiments disclosed herein may thus significantly improve the economic performance of the pyrolysis process.

While the disclosure includes a limited number of embodiments, those skilled in the art, having benefit of this disclosure, will appreciate that other embodiments may be devised which do not depart from the scope of the present disclosure. Accordingly, the scope should be limited only by the attached claims.

1. A method of manufacturing a heat exchange device having at least one heat exchange tube, comprising:
determining a peak heat flux area of the at least one heat exchange tube; and
disposing in the at least one heat exchange tube a flow enhancement device for creating a desirable flow pattern in a process fluid flowing through the at least one heat exchange tube;
wherein the flow enhancement device is disposed in the at least one heat exchange tube upstream of or at the determined peak heat flux area of the at least one heat exchange tube.

2. The method of claim 1, wherein the at least one heat exchange tube makes multiple passes, each pass having a peak heat flux area, the method comprising:
disposing in two or more of the passes of the at least one heat exchange tube a flow enhancement device for creating a desirable flow pattern in a process fluid flowing through the at least one heat exchange tube;
wherein each respective flow enhancement device is disposed in the two or more passes of the at least one heat exchange tube upstream of or at the determined peak heat flux area of the at least one heat exchange tube pass.

3. The method of claim 1 or claim 2, further comprising at least one of:
determining a length of a desirable flow pattern zone resulting from placement of the flow enhancement device in the at least one heat exchange tube; and
selecting a distance upstream of the determined peak heat flux area to dispose the flow enhancement device in the at least one heat exchange tube based on at least one of the determined length of the desirable flow pattern zone such;
determining a distance upstream of the determined peak heat flux area to maximize heat flux over the determined length of the desirable flow pattern zone; and
repeating one or more of the determining a length, selecting a distance, and determining a distance to optimize one or more of the heat flux over the length of the desirable flow pattern zone, the length of the desirable flow pattern zone, a design of the flow enhancement device, and an operating parameter of the heat exchange device.

4. The method of claim 1, wherein the flow enhancement device has a twist angle between 100° and 360°.

5. The method of claim 1, wherein the flow enhancement device divides a flow area of the heat exchange tube into two passages.

6. The method of claim 1, wherein an axial length of the flow enhancement device is in the range from about 100 mm to about 1000 mm.

7. The method of claim 1, wherein an axial length of the flow enhancement device is in the range from about 200 mm to about 500 mm.

8. The method of claim 1, wherein the flow enhancement device comprises a radiant coil insert.

9. A method of retrofitting a heat exchange device having at least one heat exchange tube, comprising:
determining a peak heat flux area of the at least one heat exchange tube; and
replacing at least a portion of the at least one heat exchange tube upstream of the determined peak heat flux area with a flow enhancement device for creating a desirable flow pattern in a process fluid flowing through the at least one heat exchange tube.

10. The method of claim 9, wherein the at least one heat exchange tube makes multiple passes through a heat transfer zone, each pass having a peak heat flux area, the method comprising:
replacing in two or more of the passes at least a portion of the at least one heat exchange tube upstream of the determined peak heat flux area with a flow enhancement device for creating a desirable flow pattern in the process fluid flowing through the at least one heat exchange tube.

11. The method of claim 9 or claim 10, further comprising at least one of:
determining a length of a desirable flow pattern zone resulting from placement of the flow enhancement device in the at least one heat exchange tube; and
selecting a distance upstream of the determined peak heat flux area to dispose the flow enhancement device in the at least one heat exchange tube based on at least one of the determined length of the desirable flow pattern zone;
determining a distance upstream of the determined peak heat flux area to maximize the heat flux over the determined length of the desirable flow pattern zone; and
repeating one or more of the determining a length, selecting a distance, and determining a distance to optimize one or more of the heat flux over the length of the turbulent zone, the length of the desirable flow pattern zone, a design of the flow enhancement device, and an operating parameter of the heat exchange device.

12. A heat exchange device, comprising:
at least one heat exchange tube; and
a flow enhancement device disposed in the at least one heat exchange tube for creating a desirable flow pattern in a process fluid flowing through the at least one heat exchange tube;
wherein the flow enhancement device is disposed in the at least one heat exchange tube upstream of or at a determined peak heat flux area of the at least one heat exchange tube.

13. The heat exchange device of claim 12, wherein the heat exchange device comprises a furnace for the heating of a pyrolysis feedstock, the furnace comprising a heating section including:
a heating chamber;
a plurality of the at least one heat exchange tubes positioned in the heating chamber; and
a plurality of burners.

14. A process for producing olefins, the process comprising:
passing a hydrocarbon through a heat exchange tube in a radiant heating chamber at conditions to effect pyrolysis of the hydrocarbon, the heat exchange tube having an flow enhancement device disposed therein for creating a desirable flow pattern of the hydrocarbon flowing through the heat exchange tube;
wherein the flow enhancement device was selectively disposed in the at least one heat exchange tube upstream of or at a determined peak heat flux area of the at least one heat exchange tube.
